(19) United States
(12) Patent Application Publication
(10) Pub. No.: US 2001/0016593 A1
(43) Pub. Date: Aug. 23, 2001

(54) Element giving rapid release of nicotine for transmucosal administration

(76) Inventor: Paul C. Wilhelmsen, Alamo, CA (US)

Correspondence Address: PAUL C. WILHELMSEN, 5100 Channel Ave., Richmond, CA, 94804-4646, US

(21) Appl. No.: 09/771,454

(22) Filed: Jan. 29, 2001

Related U.S. Application Data
(63) Continuation-in-part of application No. 09/292,045, filed on Apr. 14, 1999, now Pat. No. 6,248,760.

(51) Int. Cl. A61K 9/68

(52) U.S. Cl. 514/343; 424/440

(57) ABSTRACT

An pharmacological element, generally configured in the shape of a tablet, for the transmucosal administration of nicotine is made to effect the equivalent physiological and psychological responses of smoking a cigarette. The element is made by placing between 0.5 mg and 5 mg of nicotine in a layer upon a non-nicotine containing matrix. The nicotine containing layer rapidly dissolves by the warmth and saliva in the mouth. The nicotine is then rapidly absorbed by the intra-oral mucosal surfaces of the mouth, and then felt as a sharp pulse in the brain in a period of less than two minutes, but usually in less than 30 seconds. A multi-layered element is also provided where the nicotine is placed in a plurality of layers separated by one or more layers that are essentially free of nicotine to support the nicotine containing layers, and, to provide a time delay between the sharp pulses produced by the nicotine containing layers. The multi-layered element effects the equivalent of taking several drags of a cigarette over a period of minutes with each nicotine-containing layer in the multi-layered element effecting a pulse of nicotine similar to each drag on a cigarette. An additional benefit of enhanced mental acuity is disclosed for the nicotine elements.

### COPENDING APPLICATION

This is a Continuation-in-Part (CIP) of allowed U.S. application Ser. No. 09/292,045, filed Apr. 20, 1999.

### FEDERALLY SPONSORED RESEARCH

This application is not the subject of any federally sponsored research and development.

### BACKGROUND

1. Field of Invention

The present invention relates to an element for transmucosal administration of a medicament. Specifically, it relates to an element that will give a rapid release of nicotine.

2. Description of Prior Art

Nicotine is the most widely distributed of the plant alkaloids. It occurs in two separate phyla of the plant kingdom, Pteridophytes and Spermatophytes. Despite its wide distribution, for practical purposes, nicotine is obtained from the tabacum and rustica species of the Nicotina genus. However, it is interesting that Australian Aboriginal people chewed “pituri”, a nicotine preparation made from Duboisia hopoodi.

Nicotine can be isolated as an oily, volatile base with a sharp burning taste. Nicotine will form acid salts with most acids. Nicotine can be introduced into the body in many ways. The most popular way is by smoking cigarettes. When a cigarette is smoked, the partial oxidation of the tobacco causes the vaporizing of some of the nicotine. The nicotine vapor as well as nicotine adsorbed on partial oxidation products of the cigarette is quickly absorbed through the lungs. A dozen beats of the heart can carry nicotine from the lungs to the brain in less than 20 seconds.

Smoking cigarettes introduces many undesirable materials into the person smoking, as well as into the environment. Because of this, most smokers would like to quit smoking. However, a craving for nicotine, irritability, restlessness, difficulty in concentrating, headaches, an increase in weight, and anxiety often follow cessation of smoking. Supplying nicotine in an alternative way has been helpful in some cases of those wishing to quit smoking. Supplying nicotine without the toxic and carcinogenic substances associated with smoking is likely to reduce the hazard of nicotine use and abuse. Several alternative ways of delivering nicotine have been proposed.

Frith (Life Sciences, 1967, 6, 321-326) described experiments where tablets containing 0.1 mg of nicotine were administered orally. The subjects were instructed to chew the tablets until the tablets were dissolved. These tablets had the nicotine distributed uniformly throughout the tablets.

Lennox M. Johnston and M.D. Glasg in the Dec. 19, 1942 edition of Lancet, p.742, described intravenous injection of nicotine. They reported that after several injections, an injection was preferred to a cigarette. Intravenous injection has continued to be used in scientific experimentation. See for example Lucchesi et al. (Clin. Pharm. Ther., 1967, 8, 789-96), Soria et al. (Psychopharmacology 1996, 128, 1000-1005), Benowitz et al. (J. of Pharmacol. Exp. Ther. 1990,254, 1000-1005), and Rosenberg et al. (Clin. Pharmacol. Exp. Ther. 1980,28, 516-22). However, intravenous injection is obviously not a suitable replacement for smoking in most situations.

Jarvik et al. (Clin. Pharm. Ther. 1970,11, 574-576) described experiments where 10 mg tablets of nicotine tartrate were swallowed at two hour intervals by several subjects. The idea was that although the ionic form of nicotine would not be absorbed in the stomach, it would be absorbed in the intestine. However, most of the nicotine absorbed in the intestine is metabolized by the liver before it reaches the nervous system. The large amounts of nicotine in these experiments, as compared with about 1 mg available in a cigarette, had only a small effect. (Actually the abstract of this paper states that 10 mg of nicotine tartrate per kilogram was administered, but this is an obvious error.)

Wesnes and Warburton (Psychopharmacology, 1984, 82, 147-150) described experiments where nicotine was added to dextrose tablets. They also (Psychopharmacology, 1986, 89, 55-59) used nicotine absorbed in magnesium hydroxide pills. In their experiments they covered the taste of nicotine with hot pepper sauce. The sauce is acidic. Under the conditions of their experiments the alkaline reaction of the basic magnesium hydroxide would be useful in neutralizing the acidity of the sauce. In these experiments a dilute nicotine solution was allowed to soak into the dextrose or magnesium hydroxide pills. It is likely that the solution would be soaked into the inevitable porosity of the compacted (sintered) pill to a considerable depth. The subjects were requested to hold the pills in their mouths for 5 minutes before swallowing. The described pills did not give a rapid release of nicotine.

Shaw (U.S. Pat. No. 4,806,356, 1989) described a lozenge made by compression molding with nicotine distributed uniformly throughout. Shaw's lozenges will slowly release nicotine. Santus (U.S. Pat. No. 5,549,906, 1996) described nicotine-containing lozenges where the nicotine is uniformly distributed through a nonnutritive sweetener and an absorbent excipient. Santus discussed how the administration of nicotine is obtained by allowing the lozenge to completely dissolve in the mouth. Several nicotine lozenges have been commercialized and are available as over-the-counter products in the U.K. These contain 0.5 mg nicotine distributed uniformly throughout the lozenges.

Sahakian et al. (Brit. J. Psychiatry, 1989,154, 797-800) and Jones et al (Psychopharmacology, 1992, 108, 485-494) describe using a subcutaneous injection of nicotine. While of possible use in scientific studies, this does not fill the need for the usual smoker. Under some conditions, such an injection can cause severe muscular irritation. It was, however, found efficacious in improving discriminative sensitivity and reaction time in patients with dementia of the Alzheimer type.

The absorption of drugs such as nitroglycerin and scopolamine through the skin is well described. The absorption of nicotine through the skin has also long been known. Workers coming into physical contact with dew on tobacco leaves can develop what is called “green tobacco sickness”. See for example Gehlbach et al. (JAMA., 1974, 229, 1880-1883). The transdermal administration of nicotine has been the subject of many patents. For example, Baker et al. U.S. Pat. No. 4,839,174, 1989, U.S. Pat. No. 4,943,435, 1990, and U.S. Pat. No. 5,135,753, 1992. The commercial products based on these patents have turned out to be quite expensive. More importantly, the patches give a rather constant level of nicotine in the blood resulting in rapid desensitization. Transdermal nicotine patches have another disadvantage in that it is difficult to administer nicotine at the rate required by different subjects. Benowitz et al. (J. Pharmacol. Exp. Ther., 1982, 21, 368-373) and Feyerabend et al. (Brit. J. Clin. Pharmacol., 1985, 19, 239-249) have found that there are large variations in the rate nicotine is eliminated and receptors become resensitized by smokers. It is also advantageous for users to be able to vary dosage to cope with times of intense craving.

Nicotine chewing gum is another way of administering nicotine. Ellis et al. (U.S. Pat. No. 3,865,026, 1907) provides an early example. More recent examples are Ferno et al. (U.S. Pat. No. 3,845,217, 1974) and Lichtneckert et al.(U.S. Pat. No. 3,877,468, 1975 and U.S. Pat. No. 3,901,248, 1975). Products of these patents are now being marketed on an international scale. These products combine a nicotine- containing cation exchange resin complex in a gum base. However, chewing gum is not socially acceptable in some circumstances. Some users of nicotine gum have complained about the effort required to chew it. Many users complain about the taste.

Much has been learned in recent years. Russell (Nicotine Replacement: A Critical Evaluation: Pomerleau, O. F. and Pomerleau, C. S., eds.; Alan R. Liss, Inc.: New York 1988, 63-94) observed that cigarette smoking provides an initial sharp rise in blood nicotine level which is missing in transdermal nicotine and in nicotine gum. Henningfield et al. (Drug Alcohol Dependence, 1993, 33, 23-39) showed that the arteriovenous differences during cigarette smoking are substantial, with arterial levels exceeding venous levels six to ten fold. Benowitz (Nicotine Safety and Toxicity, Benowitz, Neal L., ed.; Oxford: New York 1998; 3-16) observed that there is an intense pharmacological response due to the high levels of nicotine entering the brain and the effects occurring rapidly before the development of desensitazation. The nicotine level in the brain declines between cigarettes as the nicotine is distributed to other body tissues. This decline in nicotine level provides an opportunity for resensitization of receptors, allowing some positive reinforcement despite the development of tolerance.

These conclusions have been verified by Stitzer and DeWit (Nicotine Safety and Toxicity, Benowitz, Neal L., ed.; Oxford: New York 1998, 119-131) where they measured the acceptance by users of an intravenous injection, a nasal spray, a vapor inhaler, nicotine gum, and a nicotine patch. A rating scale was used where the subjects gave 0 for liking “not at all” and 4 for liking “awful lot”. Both the 2 and 4 mg. nicotine gums were given negative ratings. The nasal spray, the vapor inhaler, and the nicotine patch all were given ratings of about 0. The average rating by the subjects for an injection of 1.5 mg of nicotine was 0.8 and for 3 mg the rating was 2.6. The average ratings for 1.4 mg cigarettes was 1.5 and for 2.9 mg cigarettes was 1.8. The high rating of the intravenous injection shows that a rapid increase in venous levels can give brain nicotine levels that give satisfaction to the smoker.

Pomerleau (Ann. Behav. Med. 1989, 36, 158-163) identified some of the criteria that should be met for a nicotine dosing method to be considered satisfactory: 1) the method should be safe and easy to use; 2) specified doses should be accurately and reproducibly delivered; and 3) the pharmacokinetics should resemble those of cigarette smoking, since a sharp rise in plasma nicotine followed by a gradual decay is the pattern responsible for the unique reinforcing effects of cigarette smoking.

There have been several proposals to overcome the failure of transdermal patches and nicotine gum to deliver the sharp peak in blood nicotine level that is necessary to satisfy many smokers. For example, Rose et al. (U.S. Pat. No. 5,834,011, 1998) suggested using a nasal spray to give a rapid administration of nicotine. Rose suggested further that the aerosol be combined with transdermal patches. However, the aerosol delivery system is inconvenient and unpleasant for the user, as well as being expensive.

Baker et al. (U.S. Pat. No. 5,135,753, 1992) suggested combining transdermal patches with buccal administration of nicotine. The suggested tablets have their nicotine distributed uniformly and will not give the rapid release of nicotine that is lacking in the transdermal patches.

Perfetti (U.S. Pat. No. 5,488,962, 1996) has addressed some of the problems with the present nicotine gum. Perfetti proposed elimination of the cation-exchange resin and to use lower levels of nicotine in the gum. The improved gum described by Perfetti is still not convenient for the user. The improved gum will still not give the sharp nicotine peak that is desired.

Russell et al. (Brit. Med. J, 1983, 286, 683-684) described a nasal solution that could deliver nicotine via the nose. A two- percent solution of nicotine was combined with a thickening agent. Each dose was contained in a plastic container that could be opened and squeezed to administer the nicotine. These doses have the disadvantage of being expensive to manufacture and inconvenient to use. Ferno et al. was granted U.S. Pat. No. 4,579,588, 1986 for a similar idea.

Ray et al. (U.S. Pat. No. 4,655,231, 1987) described a synthetic snuff, which consists of a powdered salt of nicotine diluted with powdered organic sugars. Snuff is inconvenient for the user and is not socially acceptable.

Pomerleau et al. (Psychopharmacology, 1992, 108, 518-526) described an intranasal aerosol delivering system. This is an excellent method of delivering nicotine in a laboratory. It is capable of giving the short, intense pulses of nicotine that are desired. However, it is inconvenient for the user and not practical in most applications outside the laboratory.

Myers et al. U.S. Pat. No. 5,871,781, 1999, described a device for making comestible units. These units might contain nicotine. However, the process for making these units is complicated and expensive. The nicotine is uniformly distributed.

Ray (U.S. Pat. No. 4,284,089, 1981) described the use of a device to vaporize nicotine. Unfortunately, nicotine vapor has a bad taste and the described device does not provide sufficient nicotine to satisfy cigarette smokers. This device is not convenient for the user and is expensive.

Volsey, II (U.S. Pat. No. 5,865,186, 1999) described a heated device that might overcome the problem that the Ray's device had in terms of the quantity of nicotine available. This device is not convenient for the user and is complicated. Keith and Snipes (U.S. Pat. No. 4,764,378, 1988) described abuccal dosage form for transmucosal administration of nicotine. Their device attaches itself within the mouth and delivers the drug over several minutes. This device has the same disadvantages as nicotine gum.

Knudsen and Rasmussen (U.S. Pat. No. 5,284,163,1994) described a device for administering a powdered or granular material containing nicotine into the oral cavity. The nicotine containing material is chewed together with gum. Their device has the same disadvantages as the nicotine gum.

Ray and Ellis patented an oral nicotine dispenser. Their U.S. Pat. No. 4,907,605, 1990, is specifically designed to slowly release nicotine. The nicotine is sorbed into polymeric materials. Ray and Ellis described the release of thirty percent of the nicotine per hour.

Rose et al. (U.S. Pat. No. 4,953,572,4, 1990) described the use of an aerosol to deliver nicotine to the oral cavity of an individual. As stated, this is an effective way of supplying nicotine to be absorbed rapidly. However, it is complicated, unpleasant for the user, and expensive.

Mondre received U.S. Pat. No. 5,035,252 in 1990. The Mondre patent covers using a dental floss containing nicotine. While it is quite likely that nicotine will be absorbed from nicotine containing dental floss, it is unlikely that cigarette smokers would be wiling to floss as often as they would smoke a cigarette.

Mascarelli patented a lollypop that contained nicotine. His U.S. Pat. No. 5,048,544, 1991, is based on the assumption that smokers would accept lollypops as socially acceptable. To actually function, the lollypop would need to be placed in the mouth for a short time. Then must the wet, sticky lollypop be removed from the mouth for minutes ,or even hours. In the event a lower concentration of nicotine is employed, the lollypop can left in the mouth but would not give the initial high dose of nicotine that has been shown to be required.

Place et al. (U.S. Pat. No. 5,147,654, 1992) described a complicated osmotic device for delivering nicotine. The device basically delivers nicotine at a constant rate over an extended time period.

Chase (U.S. Pat. No. 5,666,979, 1997) described a-cigar like carrier for nicotine. When the end of this device is chewed, the nicotine containing material is slowly released in the mouth. The nicotine is absorbed through the buccal mucous. This is complicated to manufacture and inconvenient to use.

Monte (U.S. Pat. No. 5,810,018, 1998) described a complicated procedure involving sprays containing nicotine, a sequestering agent and one or more stimulants. This procedure is difficult to implement and is expensive.

Olovson (U.S. Pat. No. 5,645,088, 1997) described a device adapted to be held between the frontal teeth and the inner surface of the lips. The peripheral part of the holder is made of a saliva-dissolvable material, which in some cases contains nicotine. This is a complicated way of getting nicotine to be absorbed in the mouth.

Dam (U.S. Pat. Nos. 5,525,35,1996 and 5,733,574, 1998) described a nicotine-containing gel. These units are designed to have a disintegration time of 5-60 minutes. During this time, they release nicotine at a relatively constant rate.

Monte (U.S. Pat. No. 5,810,018, 1998) described the use of a sequestering agent to facilitate the absorption of nicotine in the mouth when a nicotine containing solution is sprayed into the oral cavity. This procedure is inconvenient for the user and costly to manufacture.

Stanley and Haque were granted U.S. Pat. No. 5,824,334 on Oct. 20, 1998. They described a holder and a nicotine-containing tablet. Stanley's system requires the user to place the holder in his mouth like a cigarette holder whereupon the saliva dissolves the nicotine-containing tablet. Their procedure is more complicated than has been found necessary or desired. They obtain their short pulses of nicotine by physically removing the tablets from the subject's mouth. Their earlier U.S. Pat. No. 4,571,953, 1987 for a nicotine-containing lollipop is again for a removable nicotine source. It is similar to Mascarelli's patent, supra.

### SUMMARY

Accordingly, the present invention provides an improved way of delivering nicotine. The invention is carried out by employing a pharmacological element, generally in the shape of a tablet, for the transmucosal administration of nicotine to effect the equivalent physiological and psychological responses of smoking a cigarette. The element is configured in one or more nicotine containing layers of between 0.5 mg and 5 mg of nicotine each, interleaved with non-nicotine containing layers. The nicotine containing layers rapidly dissolve by the warmth and saliva in the mouth. The nicotine is then absorbed by the intra-oral mucosal surfaces of the mouth and then felt as a sharp pulse in the brain in a period of less than two minutes, but usually in less than 30 seconds. In the multi-layered configuration, the non-nicotine containing layers separating the nicotine containing layers dissolve longer in time than do the nicotine containing layers, thus ensuring a time separation between the nicotine pulses.

The invention provides a method of treating smoking dependency by simulating the smoking of a cigarette without the carcinogenic effects smoking has on the lungs of an individual. An additional benefit of enhanced mental acuity and overall well being is provided for individuals suffering from reduced mental faculties as with age.

Further benefits include a means of supplying nicotine that satisfies the needs of cigarette smokers wishing to quit smoking; providing a means of supplying nicotine to smokers under conditions where they are unable to smoke; of providing an inexpensive way of supplying nicotine; of supplying nicotine in a way that is convenient for the user, and of supplying nicotine in way that permits the user to vary the dosage to meet short-term needs.

### BRIEF DESCRIPTION OF FIGURES

FIG. 1 is a perspective view of an element according to one embodiment of the present invention.

FIG. 2 is a perspective view of another embodiment of the element according to the invention.

FIG. 3 is a perspective view of another embodiment of the element according to the invention.

FIG. 4 is a perspective view of another embodiment of the element according to the invention.

FIGS. 4A and 5B show side and top views of another embodiment of the invention.

FIGS. 6A and 6B show side and top views of another embodiment of the invention.

FIGS. 7A and 7B show side and top view of another embodiment of the invention.

FIG. 7C shows the top view of this embodiment of the invention, and FIG. 7D shows a side (section) view, with a magnified detail of a small region 74.

FIGS. 8A-8C show another embodiment of the invention wherein

FIG. 8A is a side view,

FIG. 8B a top view, and

FIG. 8C a side view at an intermediate stage in the fabrication process.

FIGS. 9A and 9B show another embodiment of the invention, wherein

FIG. 9A is a side view and

FIG. 9B a top view.

FIG. 10 shows another embodiment of the invention, as a topview.

FIGS. 11A and 11B show another embodiment of the invention wherein

FIG. 11A is a side view and

FIG. 11B a top view.

FIG. 12 shows a top view of another embodiment of the invention.

FIGS. 13A and 13B show another embodiment of the invention where

FIG. 13A shows a top view and

FIG. 13B a side view of this embodiment with a roughened surface.

FIG. 14 shows another embodiment of the invention, as a top view.

FIGS. 15A-15C show another embodiment of the invention, where

FIG. 15A is a side view in section of one multi-layered embodiment,

FIG. 15B a side view in section of another multi-layered embodiment, and

FIG. 15C a side view in section of a third multi-layered embodiment of the invention.

FIGS. 16A-16D show three-dimensional and one-dimensional embodiments of the invention wherein

FIG. 16A and 16B are two views of a randomly selected three-dimensional element (a small rock). An embodiment of the invention was produced upon a portion of this rock, and is shown in

FIG. 16C. A further embodiment of the present invention as an idealized one-dimensional element is shown in FIG. 16D.

### DETAILED DESCRIPTION OF INVENTION.

Further benefits and advantages of the invention will be apparent from a consideration of the details of the following Examples as they relate to the figures.

### EXAMPLE 1

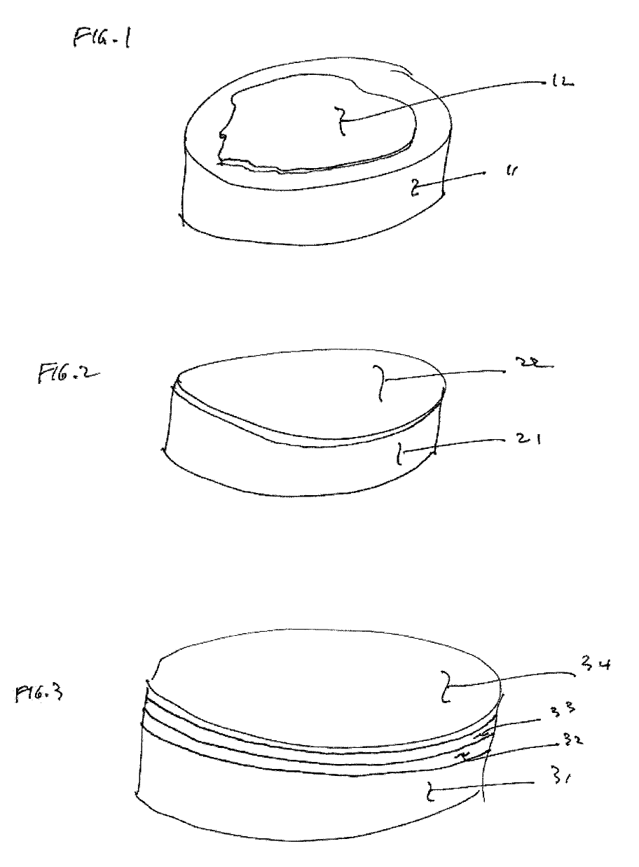

FIG. 1

A syrup was prepared by mixing 0.2004 gram of sucrose, 0.2017 gram of nicotine, and 0.2034 gram of water. As shown in FIG. 1, a thin layer 12 of this syrup was spread on top of an antacid tablet 11 with a paintbrush. The wet layer weighed 5.5 mg. The tablet was 1.7 cm in diameter and 0.4 cm thick. The total weight of the tablet was 1.5767 grams. The syrup was allowed to dry. When the tablet was placed in the mouth, the layer containing nicotine dissolved and gave a short, intense pulse of about 1.8 mg of nicotine.

### EXAMPLE 2

FIG. 2

A mixture was made from 4.0289 grams of powdered calcium carbonate, 1.0683 grams of powdered magnesium hydroxide, 1.0856 grams of caffeine, and 40.4747 gram of powdered sucrose. One gram of the above mixture was placed in the cavity of a device used to prepare tablets. The material was compressed to form a compact tablet, as shown at 21 in FIG. 2.

A second mixture was made of 0.6124 gram of nicotine tartrate and 8.0234 grams of sucrose.

The tablet-making device was opened and 0.0823 gram of the second mixture was sprinkled on top of the previously manufactured tablet. The plunger of the tablet-making device was reinserted and pressure applied to fuse the second mixture onto the previously manufactured tablet. The resulting compound tablet was removed. Nicotine containing layer 22 is above and adherent to non-nicotine layer 21. When this tablet was placed in the mouth, nicotine layer 22 dissolved to provide a short pulse of about 1 mg of the nicotine base.

The calcium carbonate and magnesium hydroxide have a basic reaction that promotes the absorption of the nicotine from nicotine tartrate. The addition of magnesium hydroxide to the calcium will reduce the tendency of the calcium to cause constipation.

### EXAMPLE 3

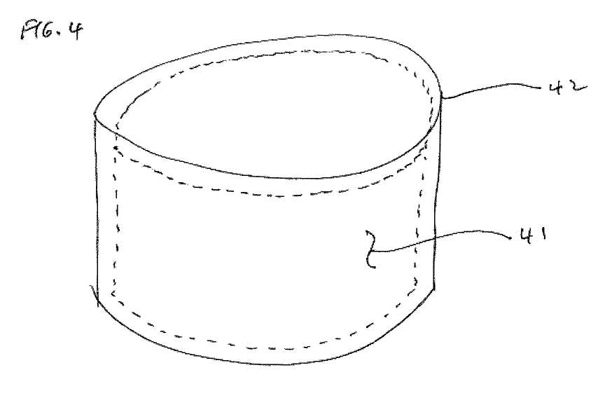

FIG. 3

One gram of the first mixture in Example 2 was placed in the cavity of a device for making tablets. The material was then compressed to a compact non-nicotine containing layer 31 of FIG. 3. The tablet- making device was opened and 0.0631 gram of the second mixture of Example 2 was sprinkled on the top of the previously made non-nicotine containing layer 31. The plunger was reinserted in the tablet-making device and pressure was used to fuse the second mixture onto the previously made non-nicotine containing layer 31 thereby forming an overlying layer 32 containing nicotine.

The plunger was removed and 0.1256 gram of the first mixture from Example 2 was sprinkled on the top of the composite tablet with layer 31 and 32. The plunger was reinserted into cavity of the tablet-making device and pressure used to fuse the new material on top of the tablet, thereby forming a overlying layer 33 which did not contain nicotine.

The plunger was removed and 0.0687 gram of the second mixture from Example 2 was sprinkled on top of the tablet. The plunger was again reinserted into the cavity of the tablet-making device and pressure applied to form an overlying, nicotine containing layer 34 and to fuse all of the layers together.

The resulting tablet thus has a first non-nicotine layer 31, a first nicotine containing layer 32, a second non-nicotine containing layer 33, and a second nicotine containing layer 34.

When placed in the mouth, the layers dissolved sequentially to provide two sharp pulses of nicotine separated by about one and a half minutes.

### EXAMPLE 4

FIG. 4

Syrup was prepared by mixing 4.0412 grams of sucrose, 1.2018 grams of nicotine tartrate, and 10.6523 grams of water. An antacid tablet was dipped into the syrup and then allowed to dry. The dried tablet is shown in FIG. 4 where the original antacid is 41 and the nicotine containing layer is 42. The original weight of the antacid tablet was 1.5465 grams. The tablet after dipping and drying weighed 1.5678 grams. When this tablet was placed in the mouth, the layer containing nicotine dissolved and gave a short, intense pulse of about 1.5 mg of nicotine.

### EXAMPLE 5

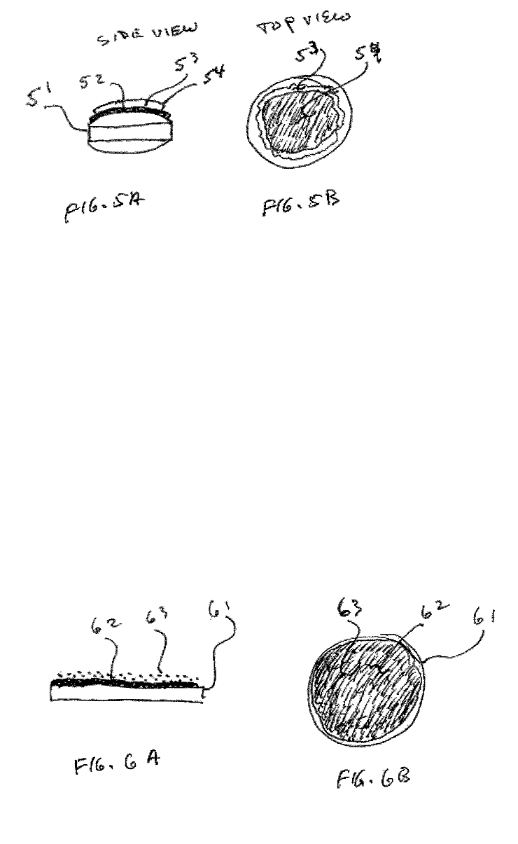

FIGS. 5A-5B

A thick paste was made by mixing 0.2134 gm of potato starch, 0.2246 gm of nicotine and 0.2107 gm of water. As shown in FIGS. 5A and 5B, a brewer's yeast tablet 51 was painted on one side 52 with a confectioner's glaze 53 made by Mantrose-Haeuser Company. This glaze was employed to prevent the nicotine paste from penetrating the brewer's yeast tablet. After the glaze had dried, the glazed brewer's yeast tablet weighed 0.7478 gm. A thin layer of the aformentioned paste 54 was painted on the glazed surface. The wet paste layer weighed 5.2 mg, of which the nicotine was calculated to be 1.7 mg. The paste was allowed to dry. When this tablet was placed in the mouth, the layer containing nicotine dissolved in a few seconds into the saliva of the mouth, and gave a short, intense nicotine pulse. Thereafter the brewer's yeast tablet along with the confectioner's glaze was swallowed.

### EXAMPLE 6

FIGS. 6A-6B

A syrup was prepared by mixing 0.2104 gm of honey, 0.2137 gm nicotine and 0.2209 gm of water. Disks were punched from polyethylene sheeting. The disks were 12 mm in diameter and 0.67 mm in thickness. One of these disks 61 was found to weigh 0.0736 gm. As shown in FIGS. 6A and 6B, a thin layer 62 of the aforementioned syrup was painted on top of the polyethylene disk 61. The wet layer was found to weigh 4.7 mg, calculated to contain 1.5 mg nicotine. The syrup was allowed to dry. The resulting film was lightly dusted with amorphous colloidal silicon dioxide 63 to reduce stickingness. When this tablet was placed in the mouth, the layer containing nicotine dissolved in a few seconds and gave a short, intense nicotine pulse. Thereafter, the polyethylene disk was spat out.

### EXAMPLE 7

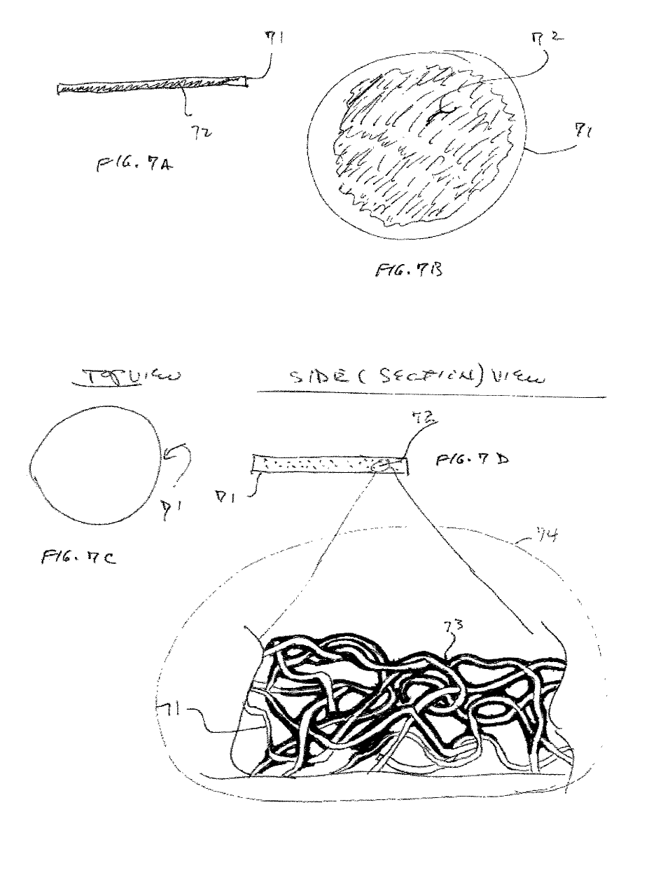

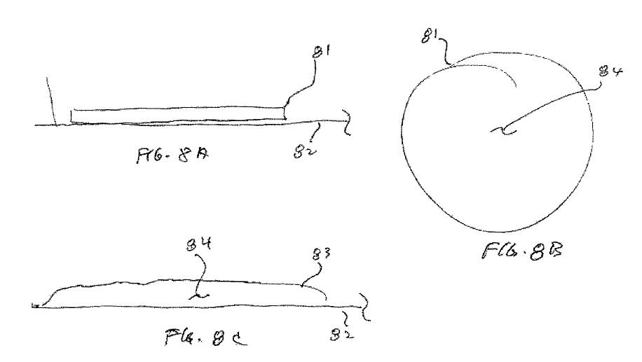

FIGS. 7A-7D

Disks were punched from Whatman #1 qualitative filter paper. The disks were 12 mm in diameter and one of them 71 as shown in FIG. 7C weighed 10.1 mg. A solution was made by adding 0.2245 gm of nicotine to 0.5326 gm of water. A 1 ml hypodermic syringe was partially filled with the nicotine solution. A small pendant drop found later to weigh 4.8 mg was made to appear on the tip of the hypodermic needle. This small drop was then transferred to the weighed filter disk 71 (FIGS. 7A and 7B). The nicotine solution 72 was absorbed on the inert (meaning inert to nicotine, its compounds, and not interfering with the administration process) cellulose fibers of the filter paper. The filter paper with its absorbed solution weighed 14.9 mg. The solution on the filter paper was allowed to dry. The result was paper fibers of which 71 (FIGS. 7D) was constituted, coated with a calculated amount of 1.4 mg of nicotine 73 and empty space (it is believed) between the coated fibers 74 (FIG. 7C). This disk when placed in the mouth absorbed saliva which solvated the nicotine which was absorbed by the tissues of the oral mucosal and/or the tongue, and gave a sharp, intense nicotine pulse. The small disk of filter paper was then swallowed.

### EXAMPLE 8

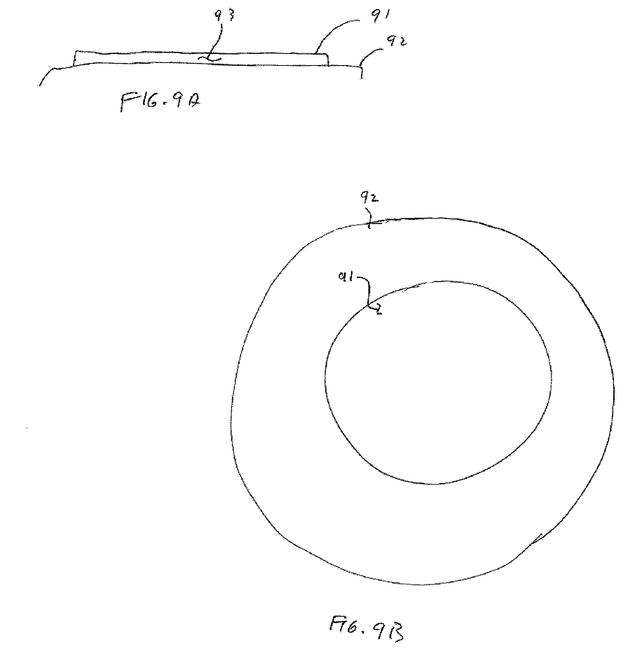

FIGS. 8A-8C

Disks were punched from “Disolvo”, “a water soluble paper” from D. Robbins & Company. The disks were 12 mm in diameter and 0.06 mm in thickness. One of these disks 81 shown in each of FIGS. 8A and 8B weighed 7.2 mg. The disk was placed on a water proof surface 82 (a polypropylene specimen cup bottom was used). A solution was made by adding 0.1552 gm of nicotine tartrate to 0.3259 gm of water. A hypodermic syringe was then used to place 14.1 mg of the nicotine tartrate solution 84, calculated to contain 1.4 mg nicotine base on the Disolvo disk 81. The somewhat swollen 83 disk containing the solution 84 (FIG. 8C) was allowed to dry on the waterproof surface 82. After drying, the paper regained its original appearance and was mechanically self-supporting. This paper disk when placed in the mouth, disintegrated in the saliva of the mouth and gave a nicotine pulse.

### EXAMPLE 9

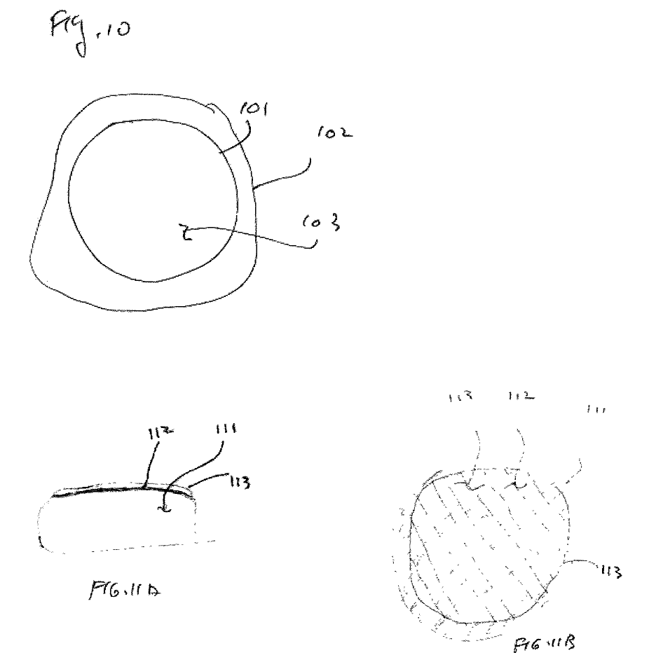

FIGS. 9A-9B

Disks were punched from “Disolvo”, “a water soluble paper” from D. Robbins & Company. The disks were 12 mm in diameter and 0.06 mm in thickness. One of these disks 91 shown in FIGS. 9A and 9B weighed 7.2 mg. The disk was placed on a waterproof surface 92 in the manner of example 8. A small amount of nicotine base was drawn into a 1 ml hypodermic syringe. A small pendant drop was made to appear on the tip of the hypodermic syringe needle. This small drop, later found to contain a calculated 1.2 mg of nicotine base, was then transferred to the weighed disk, which absorbed it (93). The disk with the added nicotine was found to weigh 8.4 mg. This paper disk, when placed in the mouth, disintegrated in the saliva of the mouth and gave a sharp, intense nicotine pulse sensation.

### EXAMPLE 10

FIG. 10

A solution of nicotine citrate was made by adding 0.1847 gm of nicotine base, 0.2047 gm of citric acid and 0.5267 gm of water. A hypodermic syringe was then used to place 9.5 mg of this solution 103, calculated to contain 1.8 mg nicotine base, on a “Disolvo” disk 101 in the manner of example 8. The disk with the nicotine citrate was allowed to dry on the water proof surface 102. This disk when placed in the mouth, disintegrated in the saliva and gave a nicotine pulse, less sharp and intense than the base.

### EXAMPLE 11

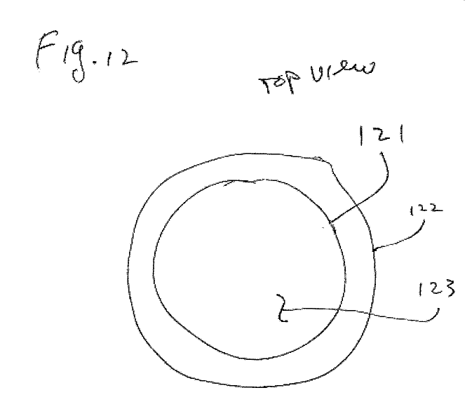

FIGS. 11A and 11B

An Altoid cinnamon flavored mint 111 was painted on one side with a confectioner's glaze 112 as shown in FIGS. 11A and 11B made by Mantrose-Haeuser Company. The glaze 112 was employed to prevent the nicotine from penetrating the confection. The glaze was allowed to dry and the mint with the dried glaze was found to weigh 0.6977 gm. A solution was made by mixing and heating 0.2678 gm of Knox unflavored gelatine and 0.5893 gm of water. The gelatine solution was allowed to cool and 0.2145 gm of nicotine base was added and dispersed uniformly throughout the gelatine solution. A thin layer 113 of the nicotine-gelatine solution was spread on the dried glaze 112. The coated tablet weighed 0.7065 gm. From the difference in tablet weights and the nicotine concentration in the coating the nicotine content was calculated to be about 1.7 mg. The gelatin layer gelled in a few minutes. When this tablet was placed in the mouth, the layer containing nicotine dissolved in the saliva in a few seconds and gave a sharp intense nicotine pulse. Thereafter the mint was allowed to dissolve in the mouth and the dissolved mint and the film of glaze were swallowed.

### EXAMPLE 12

FIG. 12

A solution of citric acid was prepared by dissolving 0.1687 gm of citric acid in 0.3323 gm of water. 0.0121 gm of the nicotine citrate described in example 10, calculated to contain 19% nicotine base, was applied to a Disolvo disk 121 on a water proof surface 122 in the manner of example 8. FIG. 12 shows the disk with the nicotine citrate solution 123 calculated to contain 2.3 mg nicotine was allowed to dry. 0.0317 gm of the above described citric acid solution, calculated to contain about 0.01 g citric acid, was then added to the disk with the previously added nicotine citrate. The disk with the added citric acid was allowed to dry. This disk when placed in the mouth disintegrated and gave a less sharp pulse of nicotine than the disk described in Example 10.

### EXAMPLE 13

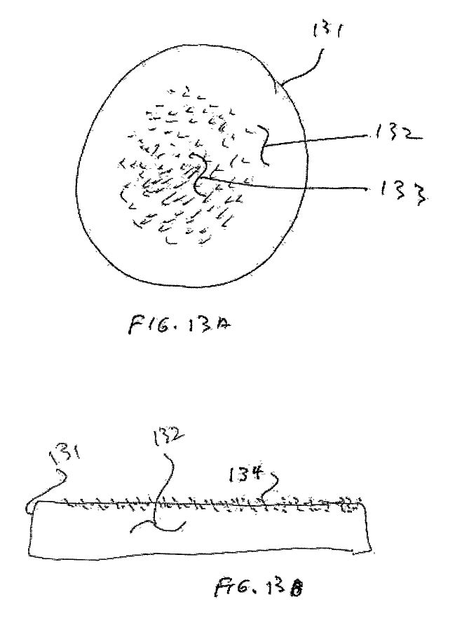

FIGS. 13A and 13B

A solution was made by adding 0.1728 gm of water to 0.2305 gm of a nicotine sulfate solution which contained 40% nicotine base. The resulting solution contained 22.9% nicotine base. 7.4 mg of this solution 132, calculated to contain 1.6 mg nicotine, was absorbed in a 12 mm disk 131 of Watman #1 filter paper (FIG. 13A). The disk was then dried. A few milligrams of sodium bicarbonate was sprinkled on the nicotine containing disk. The sodium bicarbonate was pressed into the pores of the filter paper by placing it between two sheets of 4 mil polyethylene and pressing it with small spatula. 3.6 mg of sodium bicarbonate was found to be imbedded 133 in the filter paper. The resulting disk when placed in the mouth gave a very rapid nicotine pulse, comparable to nicotine base administrations. In another embodiment of this Example, the paper surface was roughened as shown by 134 in FIG. 13B by lightly abrading it with needle-point tweezers. A quantity of sodium bicarbonate was weighed upon this surface. When smoothed out with a spatula, the fibers bound the bicarbonate powder, giving a paper surface less prone to shed bicarbonate particles, and effectively an intimate mechanical composite of nicotine-salt-impregnated or coated paper fibers surrounding powdered sodium bicarbonate.

### EXAMPLE 14

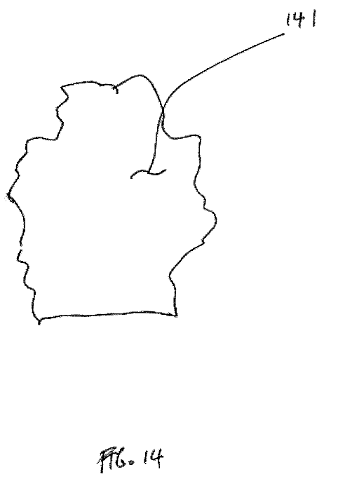

FIG. 14

An irregularly-shaped piece of Watman #1paper weighing 11.9 mg was fabricated as shown in FIG. 14. 14.3 mg of a nicotine tartrate solution containing 11.5% nicotine was absorbed into the paper which was allowed to dry. The performance of this 141 when placed in the mouth was indistinguishable from similar ones on circular disks.

### EXAMPLE 15

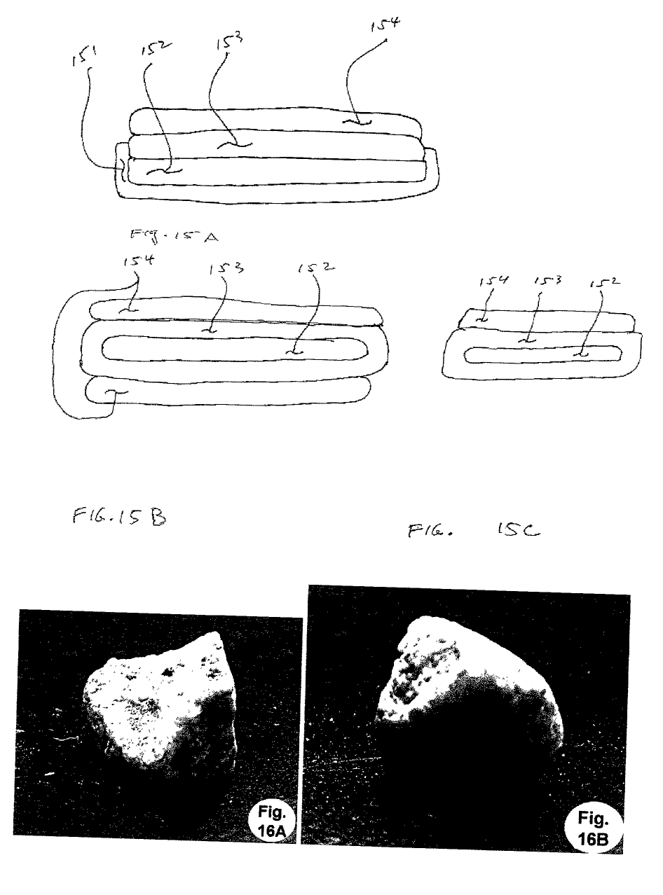

FIG. 15A

FIG. 15A shows a multi-layer embodiment of the instant invention similar as described earlier. Layers 152 and 154 are disks of nicotine containing Disolvo paper such as were used in Example 8. That is, each of these layers contain the equivalent of between 0.5 mg and 5 mg of nicotine. Bicarbonate may be incorporated therein, such as in example 13 to enhance the nicotine pulse. Dissolution of the nicotine-containing layer would take place in less than one minute, preferably less than 30 seconds. Layer 151 is a coating which does not immediately dissolve, such as the confectioner's glaze of example 11. The dissolution time of layer 151 is at least as long as the dissolution time of layer 153. Layer 153 is a slow-dissolving non-toxic layer of material. A cast sugar film such as Life-Savers™, the popular candy, would be appropriate, as would a gelatin film, such that dissolution in saliva in the mouth could be controlled to be greater in time than the nicotine containing layers, typically in the range of between one and twenty minutes. In this way, recognizable separate pulses of nicotine would be released to be experienced by the user. The spacing between nicotine pulse sensations would also be a function of the desired effect on the senses of the user. Since a single nicotine pulse has an onset time of about thirty seconds (see “Ramications” below), this would be the minimum spacing between pulses.

### EXAMPLE 15

FIGS. 15B and 15C

FIGS. 15B and 15C show the instant invention in an alternative embodiment multilayered form. Layers 152 and 154 are nicotine-containing Dissolvo paper disks, or the functional equivalent thereof As in FIG. 15A, these layers contain between 0.5 mg and 5 mg of nicotine and dissolve in less than 1 minute. Layer 153 is a slow-dissolving layer, such as a cast sugar film, that may dissolve in 1 to 20 minutes, more preferably one to five minutes. After layer 154 dissolves, a sharp pulse of nicotine is experienced. Layer 153 then dissolves within 1 to five minutes to expose nicotine layer 152 which again delivers a second sharp nicotine pulse in less than 1 minute. In FIG. 15B, nicotine-containing layer 154 is shown on each side of the slow-dissolving layer 153. Alternatively, layer 154 may completely surround layer 153. Slow-dissolving layer 153 completely surrounds nicotine layer 152 so as to ensure a time delay between the nicotine pulses experienced by the user. Layer 153 in each of these FIG. 15B embodiments is equivalent to the joining together of layers 151 and 153 in FIG. 15A. In FIG. 15C, a nicotine-containing layer 154 is on only one side of the slow-dissolving layer 153. Otherwise, the operation is the same as in FIG. 15B.

In all the multi-layer embodiments, the element when placed in the mouth, interacts with the saliva of the mouth so as to solvate each exposed layer of nicotine as the base which is absorbed by the tissues within the mouth, moves into the blood stream, where a pharmacologically effective portion of said nicotine is conveyed by the blood to the brain to thus deliver a sharp pulse thereto, equivalent to a drag on a cigarette.

### EXAMPLE 16

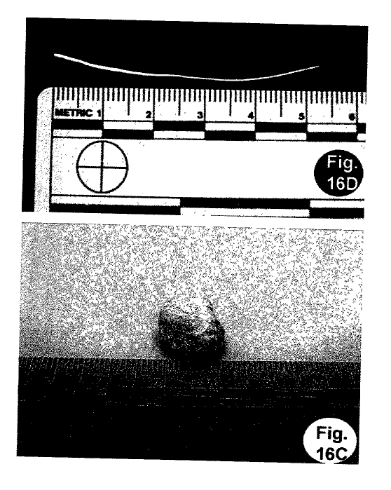

FIGS. 16A-16D

In order to determine whether flatness was a necessary restriction for the efficacy of the instant invention, a small piece of granite was selected at random from crushed rock paving. It measured about 1 cm in the largest dimension. FIGS. 16A and 16B show two views of this element. This rock was wetted by momentary immersion in tap water. A thin film of water was thus caused to exist on its surface. A piece of Dissolvo paper containing a calculated 1.7 mg of nicotine tartrate was placed in contact with the wet rock. The paper absorbed the water and underwent partial dissolution, but without much increase in volume. Much of the fibrous texture of the paper was still visible at 25× magnification. Using needle-point tweezers and adding about ten microliters of water to further soften the paper, the paper was coaxed into contact with the rock until it conformed to the arbitrary shape of the rock, and covered approximately a third the surface area. This was dried about two hours under a small desk-top lamp. The Dissolvo paper was found to be firmly attached to the rock (FIG. 16C).

When taken in the mouth, the performance of this three-dimensional element was indistinguishable from relatively flat elements.

The thin film of nicotine-dispensing material in an arbitrary three-dimensional surface shape is one example of a three-dimensional element which is capable of delivering its nicotine in one minute or less. Two-dimensional elements of regular or arbitrary shape are disclosed in examples 1-15. It logically follows that one could construct a one-dimensional element of arbitrary length, containing nicotine or a salt thereof, capable of dispensing said nicotine or a salt thereof, in less than one minute. A thin film strand of Dissolvo paper, such as that shown in FIG. 16D, is one method of realizing an essentially one-dimensional element.

### Conclusions, Ramifications, and Scope

The experiments of Examples 1-16 above describe the physiological effects of consuming a measured (calculated) dose of nicotine in the manner of the instant invention. Moreover, the inventor has found a distinct physiological and mental sensation associated with the administration of one or more nicotine doses. This is believed to strongly resemble the physiological and mental sensation associated with inhaling a puff of the smoke of burning tobacco, which it is believed have some pleasurable component or consideration thereof on the part of the recipient. There is believed to be some longer-lasting physiological and/or mental satisfaction, or lack of further nicotine effect desire, after one or more administrations of nicotine, just as is the case for most people after smoking a part or all of one or more cigarettes.

The examples as described above are of necessity reported in a subjective manner. From example 9, for instance, the taste sensation seemed to onset perhaps one second after administration, the “nicotine pulse sensation” seemed to onset about 30 seconds after administration and the physiological or mental sensation seemed to persist several minutes after administration. In these subjective terms, a sharp pulse had a faster onset than a pulse, a less sharp pulse had a slower onset, an intense pulse had a greater amplitude, and the differences were enough to be deemed worthy of comment.

Wesner and Warburton (Phermac. Ther 1983, 21, 189-208) reviewed experiment where nicotine was shown to improve mental efficiency, rapid information processing, learning, and vigilance. Jones et al (Psychopharmacology, 1992, 108, 485-494) and Sahaleian et al (Br. J Psychiatry 1989, 154, 797-800) reported that nicotine administration combated the confusion, memory loss and lack of alertness associated with Alzheimer's disease. These motivated the inventor to develop experiments using his invention for this and similar such mental conditions. Thus, for a period of over 18 months, the inventor, who is in his 70s and is experiencing symptoms of failing mental faculties, employed his inventive nicotine elements on himself to counteract such symptoms. He found that his mental acuity including memory retention increased, and an overall sense of mental awareness and well being were enhanced.

Janet Travel (Annals New York Acad. of Science, 1960 90, 13-30) described experiments where nicotine base was absorbed percutaneously much more rapidly than nicotine salts. Beckett, Gerrod and Jenner showed the bucal absorption of nicotine was very slow at pH less than 7. These results would appear to be at variance with some of the above results. However, an analysis of the techniques employed explains the differences. Travel's experiments were done with solutions of nicotine on shaved bellies of cats. It is not surprising that the percuteous absorption of cats would be different than transmucosal absorption in humans.

Beckett and Moffet (J. Pharm. Pharmac. 1968, 20, Suppl. 239S-297S) and Beckett et al (J. Pharm. Pharmac. 1972, 24, 115-120) employed a procedure where buffered solutions were prepared with measured amounts of nicotine. Several milliliters of the various buffered solutions were each placed in subjects' mouth. The solutions were held in the subject's mouth for 5 to 15-20 minutes and then expectorated. The amount of nicotine absorbed was calculated from the difference in nicotine concentration before and after. The solutions were buffered with for example 0.05 m borax. These experiments were done in such a way that the added buffer solution determined the pH in the bucal cavity. The absorption of nicotine under the conditions employed by Beckett et al would be as reported by Beckett. However, under the conditions of the examples 1 through 13 described above, the small quantity of nicotine in the thin films employed, quickly take on the pH of saliva, generally about 8, hence the rapid absorption of thin layers of nicotine salts in transmucosal applications when administered in the form of thin films.

A thin film with for example 1 mg of nicotine in the form of nicotine tartrate would have 6.16×10⁻⁶ moles of tartaric acid. This amount of tartaric acid would be neutralized by the basicity of the saliva in the localized region surrounding the point of administration. This is shown in the following experiment.

The 12 mm disks of filter paper were made by punching several layers of paper at a time. These layers of filter paper stuck together around their periphery as a result of the punching process. In the experiments described earlier, they were separated. In a further experiment, six layers that were stuck together were placed in the mouth. In 30 seconds, it was found that the six layers of filter paper had absorbed 0.1267 gm of saliva. The compressed disks were in contact with the saliva and, in addition, time was required to absorb the liquid through the several layers. A single layer would be in contact with the same amount of saliva but would not require penetration of the several layers. It will be noted that the compact disk of filter papers has almost the same absorption area as a single 12 mm disk. This shows that there is sufficient saliva in the environment of a 12 mm disk in the mouth to provide enough bicarbonate ion to free the nicotine base from its acid salt form.

The gradiently lesser response in examples 9, 10 and 12 with gradiently increasing acidity incorporated into the element further show the relationship between functional efficacy and acidity/alkalinity balance.

The University of Bristol in their web pages (http://www.bris.ac.uklDepts/Physiology/ugteach/ugindex/s1_index/gi_tut1/page6.htm and http://www.umds.ac.uk/physiology/rbm/saliva.htm) gives saliva a bicarbonate concentration of 60 mM/liter under most conditions. The net effect is that the saliva in contact with the thin film of nicotine in the above experiment has more than enough basicity to neutralize the small amount of acid that might be combined with nicotine when in the form of its salts. The absorption of nicotine salts in the form of thin films would be as expected in Beckett's experiment with pHs greater than 7. This is why Beckett got his results and why I got mine.

From the above it is evident that elements of the instant invention provide an improved way of delivering nicotine. Elements of the present invention can be made with any desired concentration of nicotine. Smokers can take such elements with decreasing concentration of nicotine and lose their addiction to nicotine and smoking, or replace the dose of nicotine normally obtained from smoking while avoiding exposure of themselves and others to the toxic and carcinogenic substances resulting from the combustion of tobacco. The availability of elements with different concentrations of nicotine (and by varying the times between administration of such elements) makes it possible for users to vary the dosage to meet short-term needs. The elements can be made very inexpensively. They are convenient for the user to use. There are no social reasons to inhibit their use. These elements do not have many of the adverse health considerations that smoking has. The elements of the present invention can be used to supply nicotine to smokers under conditions where they are unable to smoke.

While the above description contains many embodiments, these should not be considered as limitations of the invention. For example, many alternative ways are available to make rapidly dissolving layers containing nicotine. The properties of rapidly dissolving layers containing nicotine or nicotine-containing compounds will be determined by the geometry of the element, the thickness of the layers, and the materials employed. In the preferred embodiment of this invention, the layer that contains nicotine or nicotine-containing compounds will dissolve in less than two minutes. The materials other than nicotine or nicotine-containing compounds can include sugars, non-nutritive sweeteners, coloring agents, flavoring agents, effervescent agents, binders, and other compatible materials. The inventor has found that transmucosal administration of nicotine is not as efficient as smoking where equivalent amounts of nicotine are employed. This is similar to Stitzer and DeWitt where they found the same was true for intravenous injection. Therefore accordingly, there has been used larger amounts of nicotine than most previously described studies. In the preferred embodiment, the nicotine layer should contain the equivalent of from 0.5 mg to 5 mg of nicotine. In Example 1, the nicotine-containing layer was about 0.002 cm thick. The sucrose and nicotine mixture dissolved in a few seconds. In example 2 the elements were about 1.6 cm in diameter. The nicotine-containing layer was about 0.004 cm thick. The thickness of the layer and the nature of the nicotine tartrate and of the layer containing it gave a layer that required 40 seconds to dissolve.

The part of the element not containing nicotine can be anything that is acceptable to take by mouth. It can be inert as, for example, filter paper or plastic. It can be in any shape in any size, of any dimension or dimensionality, subject only to being able to be taken orally. It can contain caffeine, vitamins, flavoring materials, buffering agents, sugars, non-nutritive sweeteners, and other compatible materials.

Accordingly, the scope of the invention includes all those variations that fall within the scope of the appended claims and their legal equivalents.

I claim:

1. A nicotine containing element for transmucosal administration to an individual, said element comprising:
a) an inert, non-toxic substrate for supporting a nicotine or a nicotine salt, comprising a disintegrating, material, said material adapted to effect a rapid release of said nicotine or nicotine salt; and
b) a pharmacological dose of nicotine or nicotine salt of between 0.5 mg and 5 mg of nicotine, wherein said nicotine dose is deposited on said material and wherein when exposed to the human mouth, dissolves in less than 2 minutes.

2. The element of claim 1 wherein said material is a water soluble paper containing said nicotine pharmacological dose deposition.

3. The element of claim 1 wherein said material is a brewer's yeast tablet painted with a confectioner's glaze to inhibit absorption of said nicotine pharmacological dose deposition.

4. The element of claim 1 wherein said material is a flavored mint confection painted with a confectioner's glaze to inhibit absorption of said nicotine pharmacological dose deposition.

5. The element of claim 1 wherein said material is a combination of water soluble paper and said nicotine pharmacological dose, said resulting combination then including a predetermined addition of citric acid.

6. The element of claim 1 wherein said element is in the shape of a tablet.

7. The element of claim 1 wherein said supporting substrate for said nicotine has an arbitrary shape.

8. A nicotine containing element for transmucosal administration to an individual, said element comprising:
a) an inert, non-toxic substrate for supporting a nicotine or nicotine salt, comprising a non-disintegrating, material, said material adapted to effect rapid release of said nicotine or nicotine salt; and
b) a pharmacological dose of nicotine or nicotine salt of between 0.5 mg and 5 mg of nicotine, wherein said nicotine dose is deposited on said material and wherein when exposed to the human mouth, dissolves in less than 2 minutes.

9. The element of claim 8 wherein said material is a combination of water absorbing paper and said nicotine pharmacological dose, said resulting combination then including a predetermined addition of sodium bicarbonate sprinkled and pressed thereon.

10. The element of claim 8 wherein said non-disintegrating material is polyethylene with said nicotine pharmacological dose deposited thereon, said non-disintegrating material when exposed to said human mouth is not swallowed, but is expectorated after said nicotine dose is dissolved.

11. The element of claim 8 wherein said non-disintegrating material comprises cellulose fibers with said nicotine pharmacological dose deposited thereon, said non-disintegrating material when exposed to said human mouth and after said nicotine dose dissolves, is swallowed.

12. The element of claim 8 wherein said element is in the shape of a tablet.

13. The element of claim 8 wherein said supporting substrate for said nicotine has an arbitrary shape.

14. A method of treating dementia with the element of either claim 1 or claim 8 comprising, placing said element in the mouth of said individual, wherein said released nicotine is administered to the brain as a sharp pulse for improving mental acuity.

15. A method of treating cigarette smoking dependency with the element of either claim 1 or claim 8 comprising, placing said element in the mouth of said individual, wherein said released nicotine is administered to the brain as a sharp pulse similar to that of smoking a cigarette.

16. A multi-layered nicotine containing element for use in transmucosal administration of sequential pulses of nicotine to an individual, said multi-layered element comprising;
at least first and second nicotine containing layers of between 0.5 mg and 5 mg in concentration made to dissolve in less than one minute separated by a third non-nicotine containing layer of an inert, non-toxic material that dissolves greater in time than said first and second nicotine containing layers,
one of said nicotine containing layers surrounded by a fourth non-nicotine containing layer of a material similar to said third layer and dissolving in time no less than said third layer,
whereby separate and distinct sequential pulses of nicotine are experienced by said individual for each sequentially dissolving layer of said nicotine containing layers.

17. The multi-layered nicotine containing element of claim 17 wherein said third and fourth layers are joined as one continuous layer so as to completely surround one of said nicotine containing layers, said joined continuous layer made of a common material dissolving in time greater than said nicotine containing layers.
